(19) United States
(12) Patent Application Publication
(10) Pub. No.: US 2026/0363854 A1
(43) Pub. Date: Nov. 19, 2026

(54) PROSTHETIC VALVES AND OUTER SKIRTS THEREOF

(71) Applicant: Edwards Lifesciences Corporation, Irvine, CA (US)

(72) Inventor: Tamir S. Levi, Zikhron Yaakov (IL)

(21) Appl. No.: 19/733,163

(22) Filed: Jul. 7, 2026

Related U.S. Application Data
(63) Continuation of application No. PCT/US2025/010659, filed on Jan. 8, 2025.
(60) Provisional application No. 63/619,167, filed on Jan. 9, 2024.

(51) Int. Cl. A61F 2/24; A61F 2/00; A61F 2/844

(52) U.S. Cl. CPC A61F 2/2418; A61F 2/2436; A61F 2002/0081; A61F 2/844; A61F 2210/0076; A61F 2220/0075

(57) ABSTRACT

The present disclosure relates to prosthetic valves an outer skirts thereof. In an example, a prosthetic valve comprises a frame and an outer skirt that includes a base layer and a textured layer. A first portion of the base layer can be attached to the frame, and a first portion of the textured layer can be attached to the first portion of the base layer. The outer skirt can be movable between folded and unfolded configurations thereof, wherein a second portion of the textured layer is folded over the first portion of the textured layer in the folded configuration, and wherein the second portion of the textured layer distally extends from the first portion of the textured layer in the unfolded configuration.

### CROSS-REFERENCE TO RELATED APPLICATIONS

This application is a continuation of PCT Patent Application No. PCT/US2025/010659 filed on Jan. 8, 2025, which claims the benefit of U.S. Provisional Application No. 63/619,167, filed Jan. 9, 2024, each of which is incorporated by reference in its entirety herein.

### FIELD

The present disclosure relates to prosthetic valves, and in particular, to outer skirts of the prosthetic valves configured to move between folded and unfolded configurations.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic heart valve over a catheter and implanting it over the native malfunctioning valve, have been developed over the years.

Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, towards the native malfunctioning valve. Once the prosthetic valve is properly positioned at the desired site of implantation, it can be expanded against the surrounding anatomy, such as an annulus of a native valve, and the delivery assembly can be retrieved thereafter.

Most expandable prosthetic valves comprise a cylindrical metal frame and prosthetic leaflets mounted inside the frame. These valves can also include an outer covering or skirt disposed around an outer surface of the frame. The outer skirt can be configured to establish a seal with the native tissue when the prosthetic heart valve is placed at the implantation site.

### SUMMARY

Some types of outer skirts can include a textured layer, optionally including, for example, outwardly extending yarns, configured to encourage tissue ingrowth so as to increase paravalvular leakage sealing against the native anatomy. A textured surface exposed to a sheath through which the prosthetic valve is advanced during delivery to the site of implantation can result in increased frictional forces that can, in turn, increase the push force required to advance the prosthetic valve through the sheath. The present disclosure is directed towards prosthetic valves comprising outer skirts configured to conceal textured surfaces thereof from the surrounding environment around the prosthetic valve, during delivery of the prosthetic valve through a sheath of a delivery apparatus, for example.

According to some aspects of the disclosure, there is provided a prosthetic valve comprising a frame movable between a radially compressed state and a radially expanded state, and an outer skirt. The outer skirt comprises a textured layer defining a first surface and a second surface. The textured layer comprises a first portion extending distally from a first free end of the textured layer to an intermediate portion, and a second portion extending between the intermediate portion and a second free end of the textured layer. The outer skirt is movable between a folded configuration in which the second portion of the textured layer is folded over the first portion of the textured layer, and an unfolded configuration in which the second portion of the textured layer extends distally from the first portion of the textured layer.

In some examples, the outer skirt further comprises a base layer defining a first surface and a second surface, and comprising a first portion extending distally from a first free end of the base layer, and a second portion extending distally from the first portion of the base layer.

In some examples, the first portion of the base layer is attached to the frame.

In some examples, the first portion of the textured layer is attached to the first portion of the base layer.

In some examples, the second free end of the textured layer is oriented towards the outflow end of the frame in the folded configuration, and is oriented towards the inflow end of the frame in the unfolded configuration.

In some examples, the second free end of the textured layer is closer to the outflow end of the frame in the folded configuration than in the unfolded configuration.

In some examples, the second surface along the second portion of the textured layer is facing the first surface along the first portion of the textured layer in the folded configuration.

In some examples, the second surface along the second portion of the textured layer is oriented towards the frame in the folded configuration.

In some examples, the second surface along the second portion of the textured layer is oriented radially outwards in the unfolded configuration.

In some examples, the second surface along the second portion of the textured layer is oriented away from the frame in the unfolded configuration.

In some examples, the textured layer comprises a plurality of radially extending yarns.

In some examples, the yarns comprise first yarns extending away from the first portion of the textured layer, and second yarns extending away from the second portion of the textured layer.

In some examples, the second yarns extend towards the first yarns in the folded configuration, and extend away from the frame in the unfolded configuration.

In some examples, the first portion of the textured layer is releasably attached to the first portion of the base layer.

In some examples, the prosthetic valve further comprises at least one retaining suture and at least one pull suture, the at least one retaining suture forming a plurality of whip stitches extending around at least one pull suture and through the textured layer and the base layer, wherein the at least one pull suture is configured to be pulled through and away from the whip stitches, thereby detaching the textured layer from the base layer.

In some examples, the base layer further comprises a third portion folded over the second portion of the base layer, and a fourth portion extending between the third portion of the base layer and a second free end of the base layer.

In some examples, the base layer further comprises an inflow fold defined between the second portion and the third portion of the base layer.

In some examples, the fourth portion of the base layer is positioned radially outwards to the second portion of the textured layer in the folded configuration.

In some examples, the fourth portion of the base layer is positioned radially inwards to the second portion of the textured layer in the unfolded configuration.

In some examples, the textured layer is disposed between the first portion of the base layer and the fourth portion of the base layer in the folded configuration.

In some examples, the prosthetic valve further comprises the at least one axial suture is taut between the outflow end portion of the frame and the second end portion of the textured layer, in the folded configuration.

According to some aspects of the disclosure, there is provided a method of implanting a prosthetic valve, comprising advancing a delivery assembly that comprises a delivery apparatus carrying the prosthetic valve retained in a radially compressed state inside an outer shaft the delivery apparatus, to a native heart valve. The prosthetic valve comprises an outer skirt retained in a folded configuration thereof inside the outer shaft.

In some examples, the method further comprises deploying the prosthetic valve out of the outer shaft.

In some examples, the method further comprises positioning a second free end of a textured layer of the outer skirt distal to native leaflets of the native heart valve.

In some examples, the method further comprises moving the outer skirt to an unfolded configuration thereof by expanding the prosthetic valve such that the second free end of a textured layer is distally pushed by the native leaflets.

In some examples, the second portion of the textured layer is folded over the first portion of the textured layer in the folded configuration.

In some examples, the moving the outer skirt to the unfolded configuration comprises unfolding the textured layer.

In some examples, the textured layer further comprises a plurality of yarns extending away from the second surface of the textured layer.

In some examples, the yarns are concealed from inner walls of the outer shaft in the folded configuration.

In some examples, the moving the outer skirt to the unfolded configuration comprises exposing the yarns to the native heart valve.

In some examples, a method or device can include any of the features recited in Examples 1-82 below.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is a perspective side view of an exemplary prosthetic valve.

FIGS. 2A and 2B are cross-sectional views taken along line 2-2 of FIG. 1, in folded and unfolded configurations of an outer skirt thereof, respectively.

FIG. 3 shows an exemplary delivery apparatus carrying an exemplary prosthetic valve.

FIGS. 4A-4D show stages in an exemplary method for implantation of a prosthetic valve inside a native heart valve.

FIGS. 5A and 5B are cross-sectional views taken along line 2-2 of FIG. 1, in folded and unfolded configurations, respectively, of an outer skirt that include a textured layer releasably attached to a base layer of the outer skirt.

FIG. 6 shows a flattened side view of a portion of the prosthetic valve of FIG. 5 in the unfolded configuration of its outer skirt.

FIG. 7 a schematic cross-sectional view of a pull suture extending along a portion of the textured layer, and whip stitches extending around the pull suture and through the textured layer and the base layer.

FIG. 8A is a cross-sectional view of a prosthetic valve that includes an axial suture extending between an outflow end portion of the frame and a second end portion of the textured skirt, in a folded configuration of the outer skirt.

FIGS. 8B and 8C shows examples of unfolded configuration of the outer skirt of FIG. 8A, in a fully flipped over and partially flipped over configuration, respectively, of the second portion of the textured layer.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” “right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

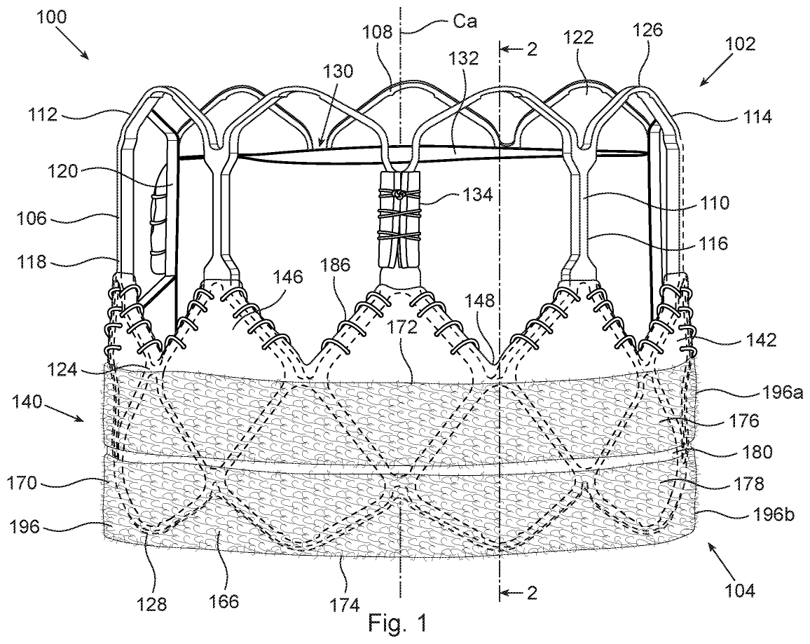

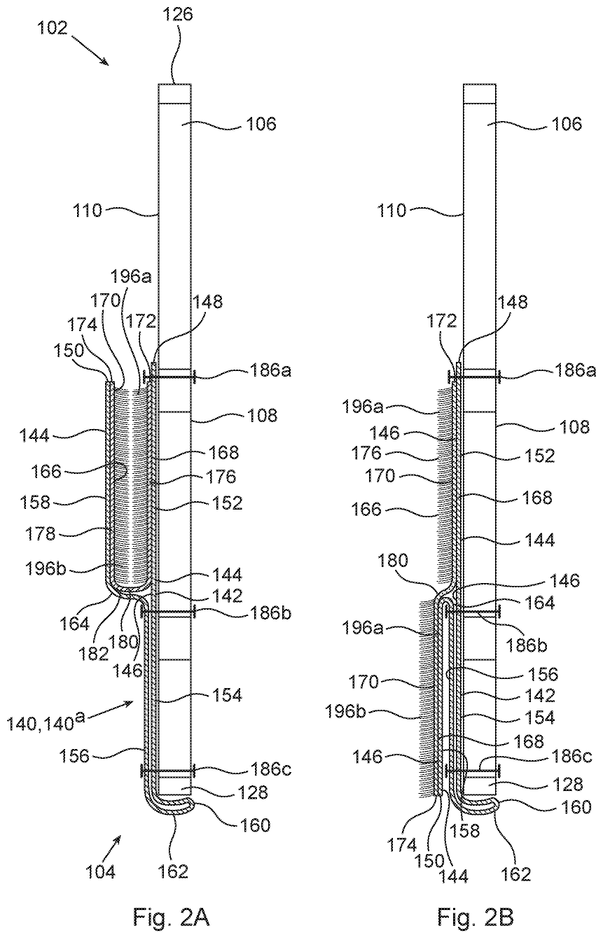

FIG. 1 shows perspective view of an example of a prosthetic valve 100. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus 12 (see FIG. 2) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve 100 of the current disclosure may include any prosthetic valve configured to be mounted within the native aortic valve, the native mitral valve, the native pulmonary valve, and the native tricuspid valve.

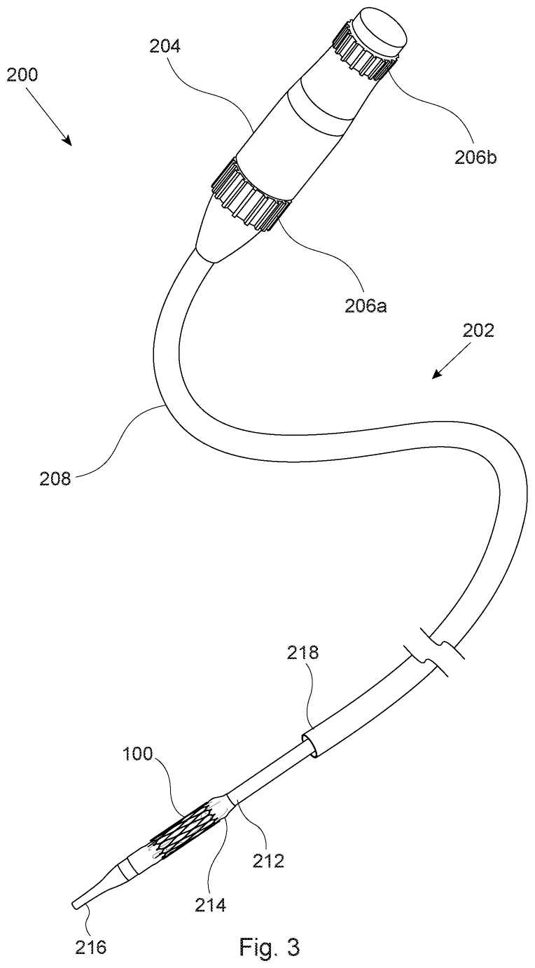

It is to be understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus 202 (see FIG. 3). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745, and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The term “plurality”, as used herein, means more than one.

The prosthetic valve 100 comprises an annular frame 106 movable between a radially compressed state and a radially expanded state, and a valvular structure 130 mounted within the frame 106. The frame extends between an inflow end 104 and an outflow end 102, and defines a central longitudinal axis Ca extending in a direction from the inflow end 104 to the outflow end 102. In some instances, the inflow end 104 is the distal end of the frame 106, and the outflow end 102 is the proximal end of the frame 106. Alternatively, depending for example on the delivery approach of the valve, the inflow end can be the proximal end of the frame, and the outflow end can be the distal end of the frame.

The term “proximal”, as used herein, generally refers to a position, direction, or portion of a device or a component of a device, which is closer to the user (for example, during an implantation procedure) and further away from the implantation site.

The term “distal”, as used herein, generally refers to a position, direction, or portion of a device or a component of a device, which is further away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

The terms “longitudinal” and “axial”, as used herein, refer to an axis extending in the proximal and distal directions, unless otherwise expressly defined.

The frame 106 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 106 can be crimped to a radially compressed state on a balloon catheter (not shown), and then expanded inside a patient by an inflatable balloon or equivalent expansion mechanism. Alternatively or additionally, the frame 106 can be made of shape-memory materials such as, but not limited to, nickel titanium alloy (e.g., Nitinol).

In the example illustrated in FIG. 1, the frame 106 is an annular, stent-like structure comprising a plurality of intersecting struts 112. In this application, the term “strut” 112 encompasses vertical struts, angled or curved struts, support posts, commissure windows, and any similar structures described by U.S. Pat. Nos. 7,993,394 and 9,393,110, which are incorporated herein by reference. A strut 112 may be any elongated member or portion of the frame 106. The frame 106 can include a plurality of strut rungs that can collectively define a plurality of cells 122 arranged in several cell rows. The frame 106 can have a cylindrical or substantially cylindrical shape having a constant diameter from the inflow end 104 to the outflow end 102 as shown, or the frame can vary in diameter along the height of the frame, as disclosed in U.S. Pat. No. 9,155,619, which is incorporated herein by reference.

Struts 112 comprise angled struts 114 and axial frame members 116. The term “axial frame member” refers to a strut or a component of the frame 106 that generally extends in an axial direction, while the term “angled strut” generally refers to a strut that can extend at an angle relative to an axial line intersecting therewith along a plane defined by the frame 106. It is to be understood that the term “angled strut” encompasses both linear angled struts and curved struts.

Two or more struts 112 can intersect at junctions 124, which can be equally or unequally spaced apart from each other. The struts 112 may be pivotable or bendable relative to each other, so as to permit frame expansion or compression. For example, the frame 106 can be formed from a single piece of material, such as a metal tube, via various processes such as, but not limited to, laser cutting, electroforming, and/or physical vapor deposition, while retaining the ability to collapse/expand radially in the absence of hinges and like.

The valvular structure 130 comprises a plurality of leaflets 132 (e.g., three leaflets), positioned at least partially within the frame 106, and configured to regulate flow of blood through the prosthetic valve 100 from the inflow end 104 to the outflow end 102. While three leaflets 132 arranged to collapse in a tricuspid arrangement, are shown in the example illustrated in FIG. 1, it will be clear that a prosthetic valve 100 can include any other number of leaflets 132. Adjacent leaflets 132 can be arranged together to form commissures 134 that are coupled (directly or indirectly) to respective portions of the frame 106, thereby securing at least a portion of the valvular structure 130 to the frame 106.

The leaflets 132 can be made from, in whole or part, biological material (e.g., pericardium), bio-compatible synthetic materials, or other such materials. Further details regarding transcatheter prosthetic heart valves, including the manner in which the valvular structure 130 can be coupled to the frame 106 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

The frame 106 can further comprise a plurality of outflow apices 126 at the outflow end 102 of the frame, and a plurality of inflow apices 128 at the inflow end 104 of the frame. A plurality of intermediate junctions 124 are disposed between the inflow end 104 and outflow end 102.

In some examples, cells 122 are coupled to adjacent cells 122 within the same row, such as within an upper-most (or proximal-most) row of cells, via axial frame members 116. Axial frame members 116 can include, in some examples, commissure windows 120 and non-commissural axial struts 118. A commissure window 120 can be configured to support a corresponding commissure 134 of the valvular structure 130. The axial frame members 116, including non-commissural axial struts 118 and commissure windows 120, can be parallel to each other and/or to the central longitudinal axis Ca of the frame 106.

While commissure windows 120 are illustrated and described herein, it is to be understood that a frame 106 can include other types of support members configured to mount a commissure 134 in any other suitable manner, such as by supporting portions of the valvular structure 130 that can be wrapped therearound, can include apertures through which sutures for attached the commissures can be passed, and the like. The terms “non-commissural axial strut” and “axial strut”, as used herein, are interchangeable, and refer to an axial frame member configured to remain unattached to the valvular structure 130. That is to say, an axial struts 118 is not configured to mount a commissure, and may be devoid of an opening such as that defined by a commissure window.

In some examples, the prosthetic valve 100 can further comprise at least one skirt or sealing member. A prosthetic valve 100 can include an outer skirt 140 disposed around an outer surface 110 of the frame 106, and configure to function, for example, as a sealing member retained between the frame 106 and the surrounding tissue of the native annulus against which the prosthetic valve is mounted, thereby reducing risk of paravalvular leakage (PVL) past the prosthetic valve 100. In some examples, a prosthetic valve can further include an inner skirt (not shown) secured to an inner surface 108 of the frame 106, and configured to function, for example, as an anchoring region for the leaflets 132 to the frame 106, and/or function to protect the leaflets 132 against damage which may be caused by contact with the frame 106, for example during valve crimping or during working cycles of the prosthetic valve 100. In some examples.

Any of an inner skirt and/or outer skirt 140 can comprise various suitable biocompatible materials, such as, but not limited to, various synthetic materials (e.g., PET) or natural tissue (e.g. pericardial tissue). In some examples, an inner skirt comprises a single sheet of material that extends continuously around the frame inner surface 108. In some examples, the outer skirt 140 can extend continuously around the frame outer surface 110.

In some examples, an outer skirt for a prosthetic valve, such as the outer skirt 140 depicted in FIG. 1, can be configured with portions that extend radially outward from the frame of the prosthetic valve (toward the surrounding native anatomy) to increase PVL sealing against the native anatomy. For example, the outer skirt 140 shown in FIG. 1 can comprise a relatively dense arrangement of outwardly extending yarns 196. However, in some instances, such configurations can increase friction of the prosthetic valve 100 against a deliver sheath through which it is delivered and advanced to deploy the prosthetic valve 100 at the site of implantation.

Increased friction between the prosthetic valve 100 and an inner surface of a delivery sheath through which it is advanced during an implantation procedure, can increase the push force is needed to push the prosthetic valve 100 through and/or out of the sheath. The push force can refer to the amount of effort or force exerted axially on the prosthetic valve 100, to distally advance the prosthetic valve 100 through a delivery sheath. In some cases, excessive push force can cause the prosthetic valve 100 to abruptly eject from the delivery sheath, causing damage to the patient.

In some examples, an outer skirt 140 can be configured to transition between a folded configuration, in which a portion of the skirt can be folded over itself to conceal the yarns 196 during delivery to the site of implantation, and an unfolded configuration, in which the yarns 196 are exposed to the surrounding anatomy upon and/or after deployment of the prosthetic valve 100.

FIG. 1 shows the outer skirt 140 in an unfolded configuration. FIGS. 2A and 2B are cross-sectional views taken along line 2-2 of FIG. 1, in folded and unfolded configurations of the outer skirt 140, respectively. The outer skirt 140 can include a base layer 142 disposed against the frame outer surface 110, and a textured layer 166 attached to the base layer 142 and separated from the frame 106 by the base layer 142.

The base layer 142 defines a first surface 144 and a second surface 146, and extends between a first free end 148 and a second free end 150 thereof. The textured layer 166 defines a first surface 168 and a second surface 170, and extends between a first free end 172 and a second free end 174 thereof. The textured layer 166 further comprises a plurality of yarns 196 extending from the second surface 170 of the textured layer 166. The plurality of yarns 196 are extending radially outward from the second surface 170 of the textured layer 166 in the unfolded configuration of the outer skirt 140, as shown, for example, in FIGS. 1 and 2B.

The first free end 148 of the base layer 142 is fixed in position relative to the frame 106 both in the folded and unfolded configurations of the outer skirt 140, while the second free end 150 of the base layer 142 is movable relative to the frame 106 and/or relative to the first free end 148, between the folded and the unfolded configurations of the outer skirt 140. In some examples, the first free end 148 of the base layer 142, which can be also referred to as an outflow end of the base layer, is closer to the outflow end 102 of the frame 106, while the second free end 150 can be closer to the inflow end 104 of the frame 106, at least in the unfolded configuration.

The first free end 172 of the textured layer 166 is fixed in position relative to the frame 106 and/or relative to the first free end 148 of the base layer 142, both in the folded and unfolded configurations of the outer skirt 140, while the second free end 174 of the textured layer 166 is movable relative to the frame 106 and/or relative to the first free end 172, between the folded and the unfolded configurations of the outer skirt 140. In some examples, the first free end 172 of the textured layer 166, which can be also referred to as an outflow end of the textured layer, is closer to the outflow end 102 of the frame 106, while the second free end 174 can be closer to the inflow end 104 of the frame 106, at least in the unfolded configuration.

Any of the base layer 142 and textured layer 166 can include, in some example, a plurality of portions which are continuous with each other along their lengths, between their respective free ends. In some examples, the base layer 142 can include a first portion 152 extending distally from the first free end 148 when the outer skirt 140 is mounted on the frame 106, a second portion 154 continuously extending from the first portion 152, a third portion 156 continuously extending from the second portion 154, and a fourth portion 158 continuously extending from the third portion 156 and terminating at the second free end 150. In some examples, the textured layer 166 can include a first portion 176 extending distally from the first free end 172 when the outer skirt 140 is mounted on the frame 106, and a second portion 178 extending between the first portion 176 and the second free end 174.

In some examples, the textured layer 166 can further include an intermediate portion 180 defined between the first portion 176 and the second portion 178 of the textured layer 166. In some examples, the yarns 196 include first yarns 196a extending away from the first portion 176 of the textured layer 166, and second yarns 196b extending away from the second portion 178 of the textured layer 166. In some examples, the intermediate portion 180, which may be significantly shorter than any of the first or second portions 176, 178 of the textured layer 166, can be devoid of yarns 196 extending away therefrom.

The base layer 142 is attached to the frame 106 such that the first surface 144 of the first portion 152, and the first surface 144 of at least part of the second portion 154, is facing, and may be optionally in direct contact with, the frame outer surface 110. In some examples, first portion 152 and at least part of the second portion 154 can be disposed flush against the frame outer surface 110.

The textured layer 166 is attached to the base layer 142 such that the first surface 168 of the first portion 176 of the textured layer 166 is facing, and may be optionally in direct contact with, the second surface 146 of the first portion 152 of the base layer 142, and such that the first surface 168 of the second portion 178 of the textured layer 166 is facing, and may be optionally in direct contact with, the second surface 146 of the fourth portion 158 of the base layer 142. In some examples, the first and second portions 176, 178 of the textured layer 166 can be adhered or bonded directly to the second surface 146 of the first and fourth portions 152, 158 of the base layer 142, respectively, such as via an adhesive or via chemical bonding (e.g., heating). In some examples, the first and second portions 176, 178 of the textured layer 166 can be sutured or coupled by other types of couplers to the first and fourth portions 152, 158 of the base layer 142, respectively.

In some examples, the axial lengths of the first and second portions 176, 178 of the textured layer 166 can be substantially equal to each other. The term “substantially equal”, when referring to axial lengths of components or portions of an outer skirt 140, refers to the axial length of one component being in the range of 85%-115% of the axial length of the other component. In some examples, the axial lengths of the first and fourth portions 152, 158 of the base layer 142 can be substantially equal to each other. In some examples, the axial lengths of the first portion 176 of the textured layer 166 and the first portion 152 of the base layer 142 are substantially equal to each other. In some examples, the axial lengths of the second portion 178 of the textured layer 166 and the fourth portion 158 of the base layer 142 are substantially equal to each other.

In some examples, the base layer 142 can be configured as an anchoring layer to which the textured layer 166 is secured. In some examples, the base layer 142 can comprise a polymeric material. In some examples, the polymeric material can be relatively thromboresistant or have a thromboresistance that is greater than that of a fabric. In some examples, the polymeric material can be relatively non-abrasive or smooth. In some examples, the polymeric material can be impermeable, such as impermeable to fluids. In some examples, the polymeric material can be partially impermeable, such as impermeable to cell migration and/or to prevent or limit neo-vascularization. In some examples, the polymeric material of the base layer 142 can comprise thermoplastic polyurethane (TPU), silicone, polyethylene terephthalate (PET), polytetrafluoroethylene (PTFE), expanded PTFE (ePTFE), and/or other similar materials. In this way, tissue ingrowth may not occur on the base layer 142 and/or propagate radially inwards. Furthermore, when an inner skirt is not included, risk of leaflets abrasion can be reduced when the base layer 142 is contacted by the leaflet 132. The base layer 142 can be, in some examples, a relatively thin layer with a thickness in a range of 10-50 μm.

In some examples, the base layer 142 can an comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide, thermoplastic polyurethane (TPU), etc.

In some examples, the textured layer 166 can include a fabric layer comprising a plurality of radially extending yarns 196. It is to be understood that the term “radially extending yarns” is not meant to limit the orientation to the yarns as being perpendicular to the central longitudinal axis Ca of the frame 106, but rather to any direction that is not parallel to the central longitudinal axis Ca. In the unfolded configuration of the outer skirt 140, the yarns 196 can be referred to as outwardly extending yarns, configured to extend away from the base layer 142 and/or the frame 106 in this configuration, as shown for example in FIGS. 1 and 2B. The terms “radially extending yarns 196”, “outwardly extending yarns 196”, and “yarns 196”, as used herein, are interchangeable, and refer to yarns extending away from the corresponding portions of the base layer 142 attached thereto, in a direction that is not parallel to the central longitudinal axis Ca, and such that the yarns extend outwardly relative to the frame 106 in the unfolded configuration of the outer skirt 140.

In some examples, the yarns 196 can be knitted or woven into and extend away from a fabric layer of the textured layer 166 which can comprise a weave of one or more fibers or yarns. In some examples, the textured layer 166 can include individual yarns 196 that that are adhered or bonded directly to the base layer 142 and extend away therefrom (e.g., via an adhesive or chemical bonding, such as via heating or another bonding method), without an additional fabric layer therebetween.

It is to be understood that the yarns 196 are shown in FIGS. 2A-2B in the form of free-ended filaments by way of illustration and not limitation, and that other forms of the yarns are similarly contemplated. In some examples, the yarns 196 can be provided in the form of loops 196, as illustrated in FIG. 1 for example. Further details on materials and/or textures of exemplary textured layers for outer skirts can be found in U.S. Patent Publications 2019/0374337 and 2023/0270544, and U.S. Patent Application No. 63/366,599, filed Jun. 17, 2022, all of which are incorporated by reference herein.

In some examples, the base layer 142 can be folded over itself at an inflow fold 160. The inflow fold 160 is defined between the second portion 154 and the third portion 156 of the base layer 142, such that the third portion 156 is laid over the second portion 154 of the base layer 142. The inflow fold 160 of the base layer 142 can be proximate to the inflow end 104 of the frame 106. In some examples, the second surface of the third portion 156 is facing, and may be optionally in direct contact with, the second surface 146 of the second portion 154 of the base layer 142. In some examples, the axial lengths of the second and third portions 154, 156 of the base layer 142 can be substantially equal to each other.

In some examples, an inflow end portion 162 of the outer skirt 140 can be folded over and/or wrapped around the inflow end 104 of the frame 106, towards the interior of the frame 106. As shown for example in FIGS. 2A-2B, an inflow end portion 162 of the base layer 142 can include the portion of the overfolded second and third portions 154, 156 extending, at the inflow end 104, from the frame outer surface 110 to the inflow fold 160. Covering the inflow end 104 of the frame 106 can advantageously protect an inner surface of a delivery sheath or catheter, through which the prosthetic valve 100 can be advanced, from being directly engaged by the inflow apices 128.

In some examples, a dividing fold 164 can be defined between the third portion 156 and the fourth portion 156 of the base layer 142, such that the third portion 156 can be defined between the inflow fold 160 and the dividing fold 164 of the base layer 142, and the fourth portion 158 can be defined between the dividing fold 164 and the second free end 150 of the base layer 142. In some examples, the first portion 152 can extend between the first free end 148 and the axial position of the dividing fold 164 of the base layer 142. In some examples, the second portion 154 can extend between the inflow fold 160 and the axial position of the dividing fold 164 of the base layer 142.

The first and second portions 152, 154 of the base layer 142 are attached to the frame by couplers, such as sutures 186, such that their position or orientation is not meant to change between the folded and unfolded configurations of the outer skirt 140. In some examples, the third portion 156 of the base layer 142 is also configured not to change its position or orientation between the folded and unfolded configurations of the outer skirt 140, optionally by being attached to the frame 106 and/or to the second portion 154 of the base layer 142. Since the first portion 176 of the textured layer 166 is attached to the first portion 152 of the base layer 142, the first portion 176 of the textured layer 166 also remains unmoved and/or does not change its orientation between the folded and unfolded configurations of the outer skirt 140.

In contrast to all other portions of the base layer 142, the fourth portion 158 of the base layer 142 is configured to move and/or change orientation between the folded and unfolded configurations of the outer skirt 140. As shown in FIGS. 2A-2B, the fourth portion 158 of the base layer 142 can be unattached to the frame 106 or to any of the other portions of the base layer 142. Since the second portion 178 of the textured layer 166 is attached to the fourth portion 158 of the base layer 142, it will move along and/or change orientation with the fourth portion 158 of the base layer 142.

In the folded configuration of the outer skirt 140, the textured layer 166 is folded over itself, as shown in FIG. 2A for example. The intermediate portion 180 defines an intermediate fold 182 of the textured layer 166 in the folded configurations, such that the first and second portions 176, 178 of the textured layer 166 extend parallel to each other, with the second surface 170 of the second portion 178 facing the second surface 170 of the first portion 176 of the textured layer 166. Since the first yarns 196a are extend away from the first portion 152 of the base layer 142, and the second yarns 196b extend away from the fourth portion 158 of the base layer 142, the folded configuration orients the first yarns 196 radially away from the frame 106, and the second yarns 196b radially towards the frame 106, such that the first yarns 196a and the second yarns 196b are oriented towards each other, concealed from the surrounding environment by the fourth portion 158 of the base layer 142.

As further shown in FIG. 2A, the fourth portion 158 of the base layer 142 extends parallel to the first portion 152 of the base layer 142 in the folded configuration. In this position, the second free end 150 of the base layer 142 and the second free end 174 of the textured layer 166 both extend towards the outflow end 102 of the frame 106. Since the fourth portion 158 is disposed radially outwards from the first portion 152 of the base layer 142, due to the thickness of the overfolded textured layer 166 disposed between the fourth and first 158, 152 portions of the base layer 142 in the folded configuration, the dividing fold 164 in this configuration is not an overfold but rather a fold of the base layer 142 extending radially relative to the frame 106, for example.

In the unfolded configuration of the outer skirt 140, the fourth portion 158 of the base layer 142 and/or the second portion 178 of the textured layer 166 extend distally from the first portion 152 of the base layer 142 and/or the first portion 176 of the textured layer 166, as shown in FIG. 2B for example. The dividing fold 164 becomes an overfold in the unfolded configuration of the outer skirt 140, with the third and fourth portion 156, 158 of the base layer 142 folded over each other about the dividing fold 164, such that the fourth portion 158 of the base layer 142 and/or the second portion 178 of the textured layer 166 extend parallel to, and/or are positioned radially away from, the second and third portions 154, 156 of the base layer 142. In this position, the second free end 150 of the base layer 142 and the second free end 174 of the textured layer 166 both extend towards the inflow end 104 of the frame 106.

As further shown in FIG. 2B, the unfolded configuration exposes the second portion 178 of the textured layer 166, such that the second surface 170 of the second portion 178 of the textured layer 166 is facing outwards, orienting the second yarns 196 outwards, away from the frame 106. Since the fourth portion 158 of the base layer 142 and/or the second portion 178 of the textured layer 166 are no longer positioned radially outwards of, and no longer conceal, the first portion 176 of the textured layer 166, upon moving from the folded to the unfolded configuration of the outer skirt 140, the first portion 176 of the textured layer 166, as well as the first yarns 196a extending outwardly therefrom, are also exposed, such that all of the yarns 196 of the textured layer 166 are exposed and extend radially outwards in the unfolded configuration.

As mentioned, fasteners or sutures, such as sutures 186 that can include whip stitches and/or in-and-out stitches, can be used to secure the base layer 142 to the frame 106. FIGS. 2A-2B illustrated exemplary outflow suture 186a configured to secure an outflow end portion of the base layer 142 to the frame 106, and a, exemplary inflow suture 186b configured to secure a lower portion of the base layer 142 to the frame 106. In some examples, an outflow suture 186a can be closer to the first free end 148 of the base layer 142, and an inflow suture 186b can be closer to the inflow end 104 of the frame 106. In some examples, the outflow suture 186a can extend through the first portion 152 of the base layer 142, as illustrated in FIGS. 2A-2B. In some examples, the outflow suture 186a can extend through both the first portion 152 of the base layer 142 and the first portion 176 of the textured layer 166. In some examples, the inflow suture 186b can extend through the second portion 154 of the base layer 142. In some examples, the inflow suture 186b can extend through both the second and the third portions 154, 156 of the base layer 142, as illustrated in FIGS. 2A-2B. It is to be understood that any number of sutures 186 or other fasteners, and positions thereof, are contemplated.

The dividing fold 164 of the base layer 142 is positioned adjacent to, and can be at least partially in contact with, the intermediate portion 180 of the textured layer 166. In some examples, the prosthetic valve 100 can further include a dividing suture 186 extending through the base layer 142 immediately below (or distal to) the dividing fold 164. As illustrated in FIGS. 2A-2B, the dividing suture 186 can pass through the upper (or proximal) end of the third portion 156 of the base layer 142, having at least part thereof, such as stitches thereof, extending radially outwards from the first surface 144 of the third portion 156. In this way, the outwardly sticking portion of the dividing suture 186b can serve as a support structure or fulcrum over which the fourth portion 158 of the base layer 142 is downwardly (or distally) folded, forming the dividing overfold 164 around the outwardly protruding part of the dividing suture 186b in the unfolded configuration of the outer skirt 140, as shown in FIG. 2B for example.

FIG. 3 shows a perspective view of an exemplary delivery assembly 200 that includes a delivery apparatus 202 adapted to deliver a prosthetic device, which can be a prosthetic valve 100 described herein. The delivery apparatus 202 can include a handle 204 and at least one catheter extending therefrom, configured to carry a prosthetic valve 100 in a crimped state through the patient's vasculature. An exemplary delivery assembly 200 comprises an exemplary delivery apparatus 202 configured to carry a balloon expandable prosthetic valve. The delivery apparatus 202 can comprise a balloon catheter 212 having an inflatable balloon 214 mounted on its distal end. A prosthetic device, such as prosthetic valve 100, can be carried in a crimped state over the balloon catheter 212.

In some examples, a delivery apparatus 202 further comprises an outer shaft 208. Optionally, an outer shaft 208 of a delivery apparatus 202 can concentrically extend over the balloon catheter 212.

The outer shaft 208 and the balloon catheter 212 can be configured to be axially movable relative to each other. For example, a proximally oriented movement of the outer shaft 208 relative to the balloon catheter 212, or a distally oriented movement of the balloon catheter 212 relative to the outer shaft 208, can expose the prosthetic valve 100 from the outer shaft 208.

A delivery apparatus 202 can further include a nosecone 216 to facilitate advancement of the delivery apparatus 202 through the patient's vasculature to the site of treatment. A nosecone shaft (concealed from view in FIG. 3) can extend proximally from the nosecone 216 through a lumen of the balloon catheter 212, towards the handle 204.

In FIG. 3, a prosthetic valve 100 is mounted on the balloon 214 and is shown in a crimped state, providing prosthetic valve 100 with a reduced diameter for delivery to the heart via the patient's vasculature. While the prosthetic valve 100 is shown in FIG. 3 as being crimped or mounted on the balloon 214 for delivery to the treatment location, it should be understood that the prosthetic valve can be crimped or mounted at a location different from the location of balloon 214 (e.g., proximal to the balloon 214) and repositioned over the balloon at some time before inflating the balloon and deploying the prosthetic valve. This off-balloon delivery allows the prosthetic valve to be crimped to a lower profile than would be possible if the prosthetic valve was crimped on top of the balloon 214. The lower profile permits the clinician to more easily navigate the delivery apparatus (including the crimped prosthetic valve) through a patient's vasculature to the treatment location. The lower profile of the crimped prosthetic valve can be particularly helpful when navigating through portions of the patient's vasculature which are particularly narrow, such as the iliac artery.

The proximal ends of the balloon catheter 212, the outer shaft 208, and/or the nosecone shaft, can be coupled to the handle 204. During delivery, the handle 204 can be maneuvered by an operator (e.g., a clinician or a surgeon) to axially advance or retract components of the delivery apparatus 202, such as the nosecone shaft, the outer shaft 208, and/or the balloon catheter 212, through the patient's vasculature and/or along the target site of implantation, as well as to inflate the balloon 214 mounted on the balloon catheter 212, for example to expand a prosthetic valve 100 mounted on the balloon 214, and to deflate the balloon 214 and retract the delivery apparatus 202, for example once the prosthetic valve 100 is mounted in the implantation site.

The handle 204 can include a steering mechanism configured to adjust the curvature of a distal end portion of the delivery apparatus 202. In the illustrated example, the handle 204 includes an adjustment member, such as the illustrated rotatable knob 206a, which in turn is operatively coupled to the proximal end portion of a pull wire (not shown). The pull wire can extend distally from the handle 204 through the outer shaft 208 and has a distal end portion affixed to the outer shaft 208 at or near the distal end of the outer shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein.

In some examples, the handle 204 can include an adjustment members such as the illustrated rotatable knob 206a, configured to adjust the axial position of the balloon catheter 212 relative to the outer shaft 208, for example for fine positioning at the implantation site. The handle can include additional knobs to control additional components of the delivery apparatus 202. Further details on the delivery apparatus 202 can be found in PCT Application No. PCT/US2021/047056, which is incorporated by reference herein.

A prosthetic valve 100 can be carried by the delivery apparatus 202 during delivery in a crimped state, and expanded, for example by balloon inflation, to secure it in a native heart valve annulus (such as an aortic annulus) or against a previously implanted prosthetic valve (for example, during valve-in-valve implantation procedures). In some examples, the balloon 214 is secured to a distal end portion of the balloon catheter 212 at its proximal end, while the balloon's distal end can be coupled, directly or indirectly, to another component of the delivery apparatus 202, such as the nosecone 216 or nosecone shaft.

Balloon 214 is configured to transition between a deflated and inflated states. Upon reaching the site of implantation, the balloon 214 can be inflated to radially expand the prosthetic valve 100. Once the prosthetic valve 100 is expanded to its functional diameter within a native annulus, the balloon 214 can be deflated, and the delivery apparatus 202 can be retrieved from the patient's body.

In some examples, the delivery apparatus 202 with the prosthetic valve 100 assembled thereon, can be packaged in a sterile package that can be supplied to end users for storage and eventual use. In some examples, when the leaflets of the prosthetic valve are made from, or include at least an inner core made from, bovine pericardium tissue or other natural or synthetic tissues, the leaflets can be treated during the manufacturing process so that they are completely or substantially dehydrated and can be stored in a partially or fully crimped state without a hydrating fluid. In this manner, the package containing the prosthetic valve and the delivery apparatus, can be free of any liquid. Methods for treating tissue leaflets for dry storage are disclosed in U.S. Pat. Nos. 8,007,992 and 8,357,387, both of which documents are incorporated herein by reference.

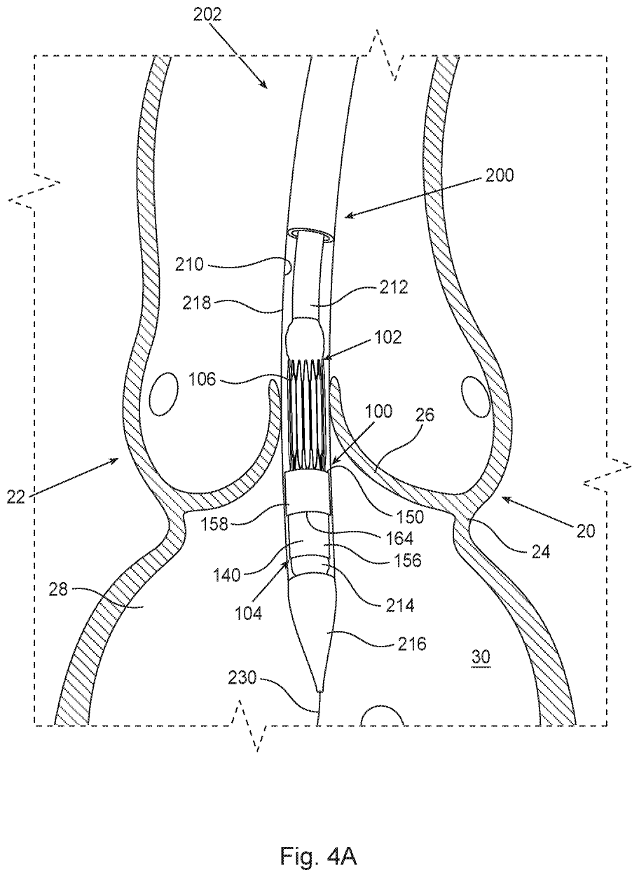

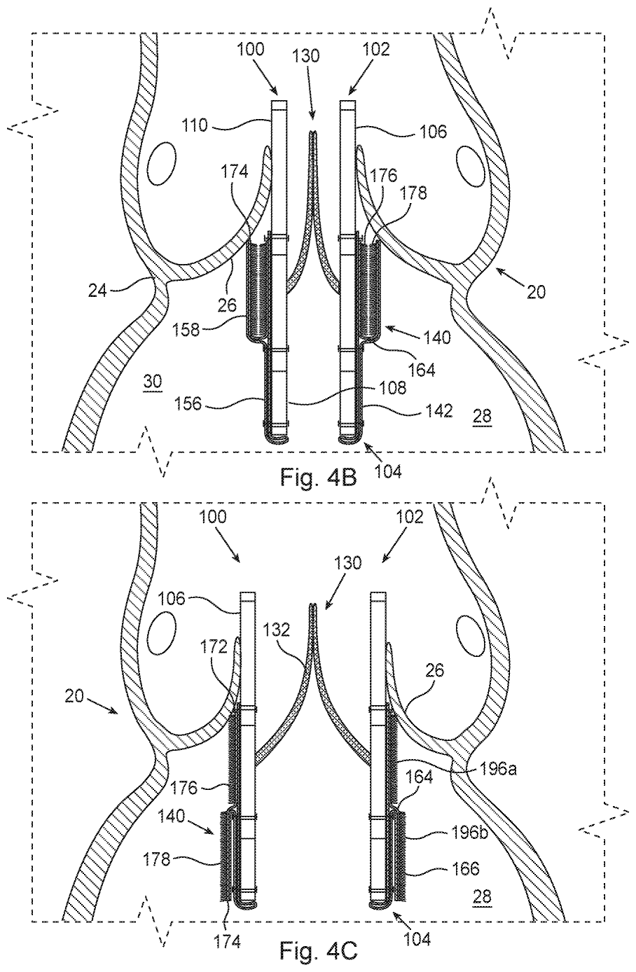

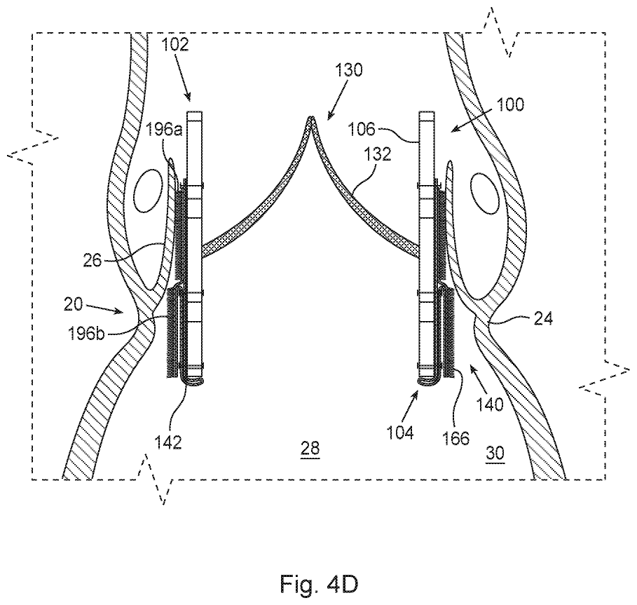

FIGS. 4A-4D show stages in an exemplary method for implantation of a prosthetic valve 100 inside a native heart valve. FIG. 4A shows a distal portion of an exemplary delivery assembly 200 advanced to the native heat valve for implantation of a prosthetic valve 100 therein. A distal portion 218 of the outer shaft 208 is shown with transparency in FIG. 4A to expose the prosthetic valve 100 situated therein. FIGS. 4B-4D show exemplary stages of prosthetic valve 100 expansion during positioning and implantation of the prosthetic valve 100 inside the native heart valve, with the delivery apparatus 202 removed from view for clarity. In the implantation procedure illustrated in FIGS. 4A-4D, the prosthetic valve 100 is implanted in a native aortic valve 20 using a transfemoral delivery approach. In other examples, a prosthetic valve 100 can be implanted at other locations (e.g., a mitral valve, a tricuspid valve, and/or a pulmonary valve), within previously-implanted prosthetic valve, and/or using other delivery approaches (e.g., transapical, transaortic, transseptal, etc.).

As shown in FIG. 4A, the delivery assembly 200, including the delivery apparatus 202 and the prosthetic valve 100 retained in a crimped state over the balloon 214, is advanced through the patient's vasculature toward the site of implantation (e.g., the aortic annulus 24) over a guidewire 230. During advancement of the delivery assembly 200, the prosthetic valve 100 can be retained inside the outer shaft 208, such as inside a distal portion 218 of the outer shaft 208, with the outer skirt 140 retained in the folded configuration, as illustrated. Prior to insertion into the patient's body, the prosthetic valve 100 can be loaded into the outer shaft 208 with the outer skirt 140 kept in the folded configuration. Once positioned inside a lumen of the outer shaft 208, the inner diameter of the outer shaft 208 can be sized to accommodate the prosthetic valve 100 with the folded configuration of the outer skirt 140, so as to maintain the outer skirt 140 in the folded configuration, without allowing it to unfold.

In the folded configuration, the textured layer 166 is completely concealed from the outer environment, such as the inner walls 210 of the outer shaft 208, maintained between the first portion 152 and the fourth portion 158 of the base layer 142. Thus, as long as the textured layer 166 is concealed between portions 152 and 158 of the base layer 142 in the folded configuration inside the outer shaft 208, the yarns 196 remain concealed as well, without any portion of the yarns 196 being exposed to the environment surrounding the prosthetic valve 100.

In some examples, as shown in FIG. 4A, the prosthetic valve 100 can be passed through the native leaflets 26 after entering the native aortic valve 20 through the aortic arch, positioning the inflow end 104 past the aortic annulus 24 and in the left ventricle 28, such as in the left ventricle outflow tract (LVOT) 30. When the prosthetic valve 100 is deployed out of the outer shaft 208, such as by distally pushing the prosthetic valve relative to the outer shaft 208 and/or by proximally pulling the outer shaft 208 relative to the prosthetic valve 100, the surface of the outer skirt 140 facing and/or being in contact with the inner walls 210 of the outer shaft 208 is the first surface 144 along the fourth portion 158 and the third portion 156 of the base layer 142.

Since the fourth portion 158 is more distanced radially outwards from the frame 106 in the folded configuration, the first surface 144 of the fourth portion 158 of the base layer 142 can be in direct contact (for example, dur to being pressed against) the inner walls 210 of the outer shaft 208, while a gap can be formed, in some examples, between the third portion 156 of the base layer 142 and the inner walls 210 of the outer shaft 208.

The first surface 144 of the base layer 142 can be a relatively smooth surface, such that frictional forces developed between the outer skirt 140 and the inner walls 210 of the outer shaft 208 during axial movement of the prosthetic valve 100 relative to the outer shaft 208, for example during deployment of the prosthetic valve 100, can be relatively modest, significantly smaller than frictional forces that could be otherwise developed therebetween if a textured surface of the textured layer 166, such as that including a plurality of yarns 196, would be in direct contact with the inner walls 210 of the outer shaft 208. As a result, push forces felt by a user advancing the prosthetic valve 100 through a guide sheath and/or outer shaft 208.

Since the drag forces applied by the relative movement between the outer shaft 208 and the prosthetic valve 100 during deployment out of the outer shaft 208 are in a direction that strives to further pull the fourth portion 158 of the base layer 142 in the proximal direction, the outer skirt 140 is maintained in its folded configuration as the prosthetic valve 100 is deployed out of the outer shaft 208.

FIG. 4B shows the prosthetic valve 100 after being deployed out of the outer shaft 208, while the prosthetic valve 100 can be still in a compressed state around the balloon 214 (which is removed from view in FIG. 4B for clarity). The delivery assembly 200 can be maneuvered to position the prosthetic valve 100, for example when exposed out of the outer shaft 208, at a desired position, such that the outer skirt 140 is positioned distal to the annulus 24 and/or the native leaflet 26. In the example illustrated in FIG. 4B, the prosthetic valve 100 is shown in its crimped state, with the outer skirt 140 still maintained in the folded configuration, positioned such that the second free end 150 of the base layer 142 and/or second free end 174 of the textured layer 166 are distal to the native leaflets 26, facing a distally-oriented surface of the leaflets 26.

In some examples, the second free end 150 of the base layer 142 and/or second free end 174 of the textured layer 166 can be brought into close proximity to, or even be brought to contact with, the distally-oriented surface of the leaflets 26, as shown in FIG. 4B. For example, the prosthetic valve 100 can be optionally deployed deeper in the left ventricle 28. Such that the second free ends 150 and 174 of the outer skirt 140 are distally distanced from the native annulus 24 and/or leaflets 26. Since the prosthetic valve 100 is still crimped over the balloon 214 in this stage, the balloon catheter can be axially maneuvered, such as by being proximally pulled, so as to bring the second free ends 150 and 174 of the outer skirt 140 closer to, and optionally up to contact with, the native leaflets 26.

Positioning of the prosthetic valve 100 at a desired position, relative to the native annulus 24, can be accomplished by one or more markers, such as a radiopaque marker (not shown) on the outer skirt 140, or taking advantage of the radiopacity of metallic components such as the frame 106 enabling visibility of end portions thereof, such as the inflow end 104, tracked by fluoroscopy of other suitable imaging modalities during the procedure, wherein the delivery apparatus 202 can be maneuvered by the clinician to align the marker or other radiopaque component with the native annulus 24, the leaflet 26, or other anatomical structures.

When a desired position of the crimped valve is achieved, the clinician can inflate the balloon 214 to facilitate expansion of the prosthetic valve 100 against the native annulus 24. During prosthetic valve expansion, the frame foreshortens in a manner that approximates the inflow end 104 and outflow end 102 to each other. In many cases, the inflow end 104 is the end that moves to a greater extent in the proximal direction. Thus, when the second free ends 150 and 174 of the outer skirt layers 142 and 166, respectively, are placed in close proximity to the native leaflets 26, as shown in FIG. 4A, expansion of the prosthetic valve 100 will result in proximal movement of the outer skirt 140 dur to valve foreshortening, pressing the second free ends 150 and 174 against the leaflets 26 in a manner that will unfold the outer skirt 140 and cause the fourth portion 158 of the base layer 142 and the second portion 178 of the textured layer 166 attached thereto, to “fall over” distally, over the second and third portions 154, 146 of the base layer 142, moving the outer skirt 140 to the unfolded configuration as shown in FIG. 4C, thereby exposing the textured layer 166 to the surrounding environment, with all of the yarns 196 oriented outwards, extending away from the frame 106.

Continued inflation of the balloon 214 will expand the prosthetic valve 100 to its final diameter, as shown in FIG. 4D, with the textured layer 166 of the outer skirt 140 pressed against the native annulus 24, after which the balloon 214 can be deflated and the delivery apparatus 202 can be retrieved from the patient's body.

Various exemplary implementations for prosthetic valves 100 and/or components thereof, such as outer skirts 140, can be referred to, throughout the specification, with superscripts, for ease of explanation of features that refer to such exemplary implementations. It is to be understood, however, that any reference to structural or functional features of any apparatus, assembly or component, without a superscript, refer to these features being commonly shared by all specific exemplary implementations that can be also indicated by superscripts. In contrast, features emphasized with respect to an exemplary implementation of device or component, including prosthetic valves 100 and/or outer skirts 140 thereof, referred to with a superscript, may be optionally shared by some but not necessarily all other exemplary implementations.

An exemplary outer skirt 140^{a}, indicated for example in FIGS. 2A-2B, can be structurally and functionally similar to any example of an outer skirt 140 described above, except that the textured layer 166 of an outer skirt 140^{a} can be non-releasably attached to the base layer 142, such as by being adhered, bonded, or non-removably sutured thereto.

A prosthetic valve can be operational inside of the patient's heart, replacing the function of the native heart valve, for the useful life of the prosthetic valve, which typically is about 15 to 25 years. In some patients, it may become desirable to remove or explant the prosthetic valve if the valve malfunctions, experiences wear and tear or deterioration from use over time, and/or as advances in prosthetic implant technologies are developed and an improved prosthetic valve device is desired. During explant procedures, the older prosthetic valve is removed such that a new prosthetic heart valve can be implanted within the diseased native valve. However, explantation of conventional prosthetic valves can be quite challenging due to the neointimal tissue formed between the outer skirt and the native annulus. Typically, the surgeon is required to cut the tissue surrounding the prosthetic valve in order to remove the prosthetic valve from the patient.

The outer skirt typically is bounded on one side by the frame of the prosthetic valve and on other side by the native annulus, leaving little room for the surgeon to access the tissue that needs to be cut from the prosthetic valve. As can be appreciated, this is a delicate procedure and may involve significant risks to the patient. In some cases, for example, explantation of a prosthetic valve can result in trauma to the tissue of the left ventricular outflow tract.

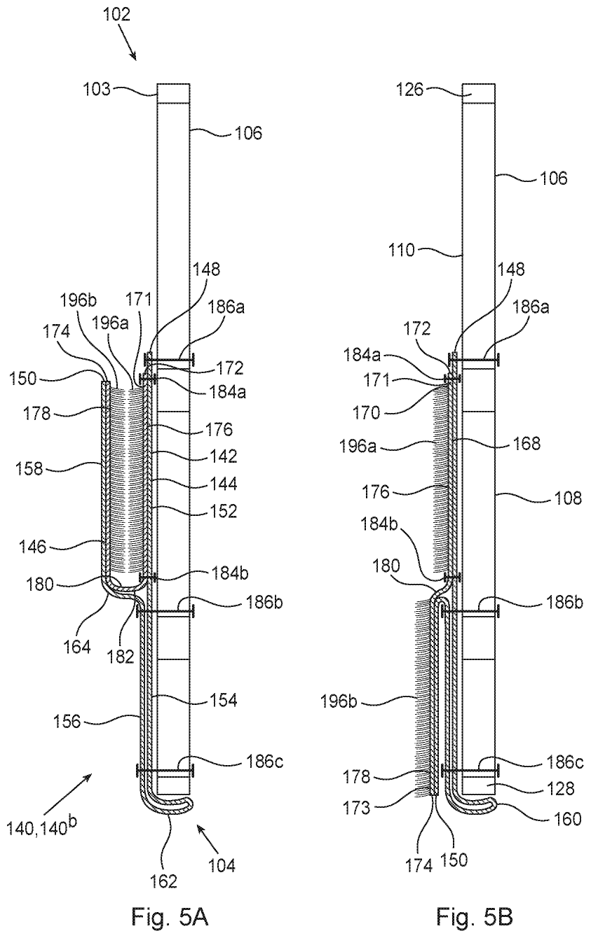

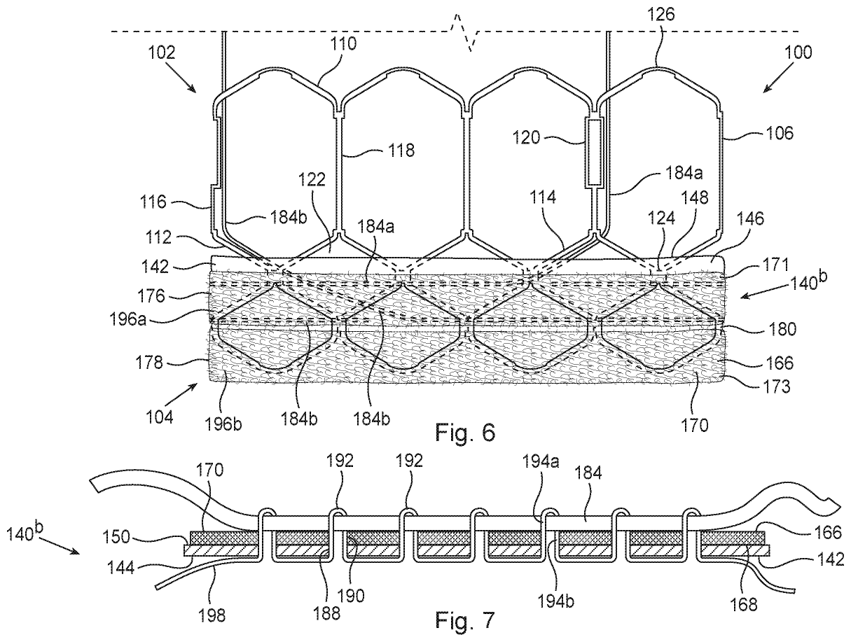

FIGS. 5A and 5B are cross-sectional views, taken along line 2-2 of FIG. 1, of a prosthetic valve 100 that includes an exemplary outer skirt 140^{b}, in folded and unfolded configurations, respectively. FIG. 6 shows a flattened view of a portion of the prosthetic valve 100 including the outer skirt 140^{b} in the unfolded configuration of FIG. 5B. Outer skirt 140^{b} can be structurally and functionally similar to any example of an outer skirt 140 described above, except that the textured layer 166 of an outer skirt 140^{a} is releasably attached to the base layer 142 via one or more pull sutures 184. As a result, a prosthetic valve 100 to which the outer skirt 140^{b} is attached can effectively seal with the native anatomy at the implantation site, while also being more easily removed (explanted) from the native anatomy. FIG. 7 a schematic cross-sectional view of the outer skirt 140^{b} of FIG. 6, showing a pull suture 184 extending along a portion of the textured layer 166 and whip stitches 192 extending around the pull suture 184 and through the textured layer 166 and the base layer 142.

The base layer 142 of outer skirt 140^{b} can be attached to the frame 106 according to any of the examples described above, including by one or more connectors, such as sutures 186. However, the textured layer 166 of the outer skirt 140^{b} can be releasably attached to the base layer 142 by a plurality of whip stitches 192 and a pull suture 184. As a result, during an explanation procedure, the textured layer 166 can be released from the base layer 142 (and the rest of the prosthetic valve 100, including the frame 106), and thus the prosthetic valve 100 can be more easily removed from the implantation site (since the textured layer 166 may have tissue adhering or growing thereto).

In some examples, more than one pull suture 184 can be used to releasably attach the textured layer 166 to the base layer 142. In the example illustrated in FIGS. 5A-6, two pull sutures 184 are shown, including an first pull suture 184a circumferentially extending along an first end portion 171 of the textured layer 166, closer to the first free end 172, and a second pull suture 184b circumferentially extending along a lower portion of the first portion 176 of the textured layer 166, closer to or at the intermediate portion 180. It is to be understood that in some examples, a single pull-suture 184, or more than two pull-sutures 184, can be used to releasably couple the textured layer 166 to the base layer 142. FIG. 7 is a cross-sectional view of the outer skirt 140^{b} along a section of a pull suture 184, which can be representative of either the first pull suture 184a, the second pull suture 184b, or any other pull suture 184 that can be used to releasably couple the textured layer 166 to the base layer 142.

As shown in more detail in the schematic cross-sectional view of FIG. 7, a plurality of whip stitches 192 can extend around the pull suture 184 and through the textured layer 166 and the base layer 142. For example, as shown in FIG. 7, the first surface of the textured layer 166 contacts or is disposed against the second surface 146 of the base layer 142. In some examples, the pull suture 184 can contact or be disposed against the second surface 170 of the textured layer 166, such as along the first portion 176.

The whip stitches 192 of a retaining suture 198 can extend through the material of the base layer 142 (e.g., passing through and being disposed within a plurality of apertures 188 in the material of the base layer 142), through the material of the textured layer 166 (e.g., passing through and being disposed within a plurality of apertures 190 in the material of the textured layer 166), and around the pull suture 184 in a whip stitch pattern along the length of an end portion of the first portion 176 of the textured layer 166. In some examples, the pull suture 184 can pass through and be disposed within a plurality of pre-formed apertures 188 in the base layer 142 and/or pre-formed apertures 190 in the textured layer 166.

Each whip stitch 192 includes a leading end portion 194a that extends through the material of the base layer 142, or alternatively through an aperture 188 in the base layer 142, outwardly through an aperture 190 in the textured layer 166, around the pull suture 184, and transitions to a trailing end portion 194b that extends inwardly through the same aperture 190 in the textured layer 166, and through a different aperture 188 in the base layer 142 (or through the material of the base layer 142), where the trailing end portion 194b transitions to the leading end portion of the next whip stitch 192 (as shown in FIG. 7). In this manner, each whip stitch 192 is threaded through a single opening or aperture 190 in the textured layer 166 and two different, but adjacent, apertures 188 in the base layer 142.

As can be seen in FIG. 7, the pull suture 184 prevents the whip stitches 192 from being pulled through the apertures 188, 190 when the pull suture 184 is positioned along the stitch line across an end portion of the first portion 176 of the textured layer 166. Therefore, the textured layer 166 can be secured to the base layer 142, which is secured to the frame 106, via a force exerted by the whip stitches 192 on the pull suture 184 and the first surface 144 of the base layer 142 when the pull suture is disposed through the stitch line and across an end portion of the first portion 176 of the textured layer 166.

In some examples, the apertures 190 in the material of the textured layer 166 and/or the apertures 188 in the material of the base layer 142 can be pre-formed, such as by laser drilling, cutting, stamping or other suitable techniques known in the art. Alternatively, the apertures 190 in the material of the textured layer 166 and/or the apertures 188 in the material of the base layer 142 can be formed as the whip stitches 192 are stitched through the material of the textured layer 166 and/or the material of the base layer 142.

When the pull suture 184 is removed (e.g., via pulling on one of a loose end of the pull suture 184 in a direction parallel to the stitch line or outwardly from the frame 106), the whip stitches 192 are no longer retained by the pull suture 184. Thus, as the textured layer 166 is pulled away from the base layer 142 and the frame 106 (or the base layer 142 and the frame 106 are pulled away from the textured layer 166), the whip stitches 192 can be withdrawn through the apertures 190 of the textured layer 166, thereby resulting in the textured layer 166 separating (or detaching or uncoupling) from the base layer 142 and the whip stitches 192 remaining attached to the base layer 142.

Since the base layer 142 can comprise a thromboresistant polymer (or a polymeric material having a greater thromboresistance than fabric material of the textured layer 166), the base layer 142 can resist tissue ingrowth and allow the base layer 142 to be more easily separated from the textured layer 166 and the tissue at the implantation site.

In some examples, the retaining suture 198 of the whip stitches 192 can be a thinner fiber, yarn, or suture relative to the thicker pull suture 184. In some examples, the retaining suture 198 can be comprised of a thinner, high tensile strength biocompatible material, such as an ultra-high molecular weight polyethylene (UHMPE) force fiber® or other similar material or combinations thereof. In some examples, the retaining suture 198 can be formed from UHMPE force fiber and can be sufficiently thin and resistant to force such that the whip stitches 192 can cut through overgrown tissue on the second surface 170 and/or yarns 196 of the textured layer 166 as the base layer 142 is separated from the textured layer 166.

In some examples, the pull suture 184 can be comprised of a thicker, high tensile strength, biocompatible material, such as a monofilament comprised of polypropylene (e.g., Prolene 4-0), polytetrafluoroethylene (PTFE), expanded PTFE (ePTFE), or other similar materials or combinations thereof. In some examples, the pull suture 184 can be a microfilament comprised of Prolene, which is configured to readily separate from or be pulled away from any surrounding ingrown tissue when pulled on by the surgeon during an explant procedure.

In some examples, the pull suture 184 can have a distinct coloration or a colored coating (e.g., a green, black, or blue coloration or coating), can be radiopaque, and/or can be otherwise tagged such that it can be identified by a surgeon during an explant procedure. In some examples, an entire length of the pull suture 184 can, for example, be comprised of a brightly colored material or have a brightly colored coating. In some examples, a portion of the pull suture 194, such as s free or loose end portion of the pull suture 194, can be comprised of a colored material, have a brightly colored coating, and/or can include a brightly colored tag or flap attached thereto.

In some examples, the pull suture 184 or a portion thereof can include a material, coating, or tag that can be visualized using a specialized visualization apparatus. For example, the pull suture 184 can comprise a radiopaque material or one or more radiopaque markers that can be visualized via a fluoroscopy or x-ray device. In some examples, the one or more radiopaque markers can be embedded within or attached to an outer surface of the pull suture 184.

In some examples, a loose end of the pull suture 184 can be tied to a commissure 134 of the prosthetic valve 100. As such, during explanation of the prosthetic valve 100, the loose end of the pull suture 184 can easily and quickly be located by a clinician and pulled, thereby pulling the pull suture 184 away from the outer skirt 140^{b} and detaching the textured layer 166 from the base layer 142.

In some examples, when the first portion 176 of the textured layer 166 is releasably coupled to the first portion 152 of the base layer 142, the second portion 178 of the textured layer 166 can remain unattached or unbonded to the base layer 142, such as to its fourth portion 158. In some examples, when the first portion 176 of the textured layer 166 is releasably coupled to the first portion 152 of the base layer 142, the second portion 178 of the textured layer 166 can be also releasably attached to the fourth portion 158 of the base layer 142, such as by one or more pull sutures 184 that can circumferentially extend around the second portion 178 of the textured layer 166.

In some cases, when a conventional outer skirt, which is not necessarily configured to move between folded and unfolded configurations, interacts with native leaflets during prosthetic valve expansion, the drag forces developed between the outer skirt and the native leaflets, optionally dragging an outflow portion of the outer leaflets in a distally-oriented direction (i.e., towards the inflow end of the frame) during frame foreshortening for example, can result in folds or ripples formed along the outer skirt, and move the outflow end of the outer skirt distally such that a shorter portion of the outer skirt remains pressed against the annulus.

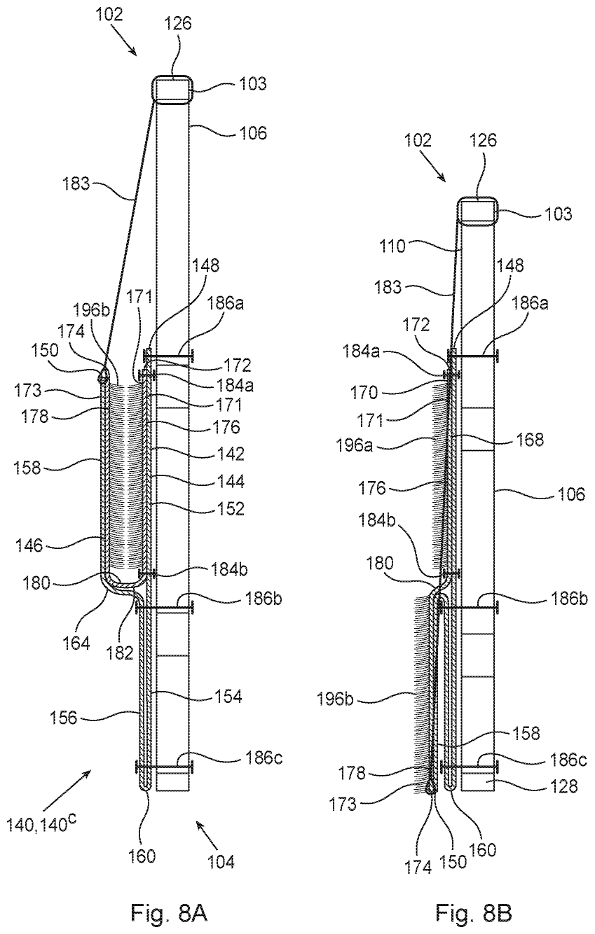

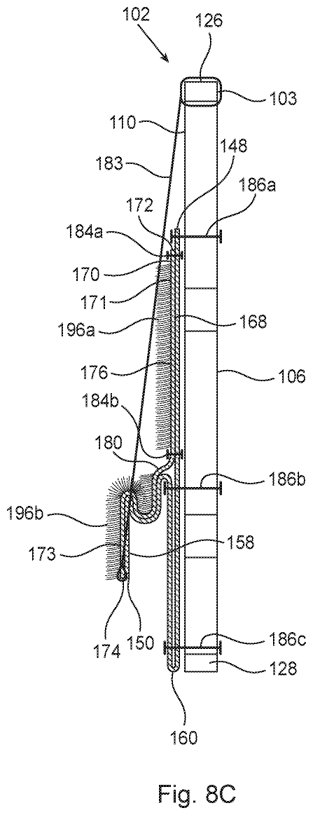

FIG. 8A is a cross-sectional view of a prosthetic valve 100 that includes an exemplary outer skirt 140^{c}, in a folded configuration. FIGS. 8B and 8C show the skirt 140^{b} in various exemplary unfolded configurations. A prosthetic valve 100 that includes an outer skirt 140^{c} can be structurally and functionally similar to any example of a prosthetic valve 100 and outer skirt 140 described above, except that the prosthetic valve 100 shown in FIGS. 8A-8C can further include one or more axial sutures 183, connecting second end portion 173 of the second portion 178 of the textured skirt 166, proximate the second free end 174, to an outflow end portion 103 of the frame 106. The axial length of the portions of the axial sutures 183 extending between their connection points to the second end portion 173 of the textured skirt 166 and the outflow end portion 103 of the frame 106 can be designed to maintain the outer skirt 140 in the folded configuration, with the axial sutures kept taut between the second end portion 173 of the textured skirt 166 and the outflow end portion 103 of the frame 106, as long as the frame 106 is in its crimped state, as shown in FIG. 8A. This serves to maintain the outer skirt 140 in the folded configuration, and prevent unintentional unfolding of the texture portion 166, even when the prosthetic valve is exposed out of the outer shaft 208, for example during maneuvering the delivery assembly 200 to reposition the prosthetic valve 100 relative to the native anatomy.

As the prosthetic valve is expanded, for example by inflating a balloon 214, foreshortening of the frame 106 approximates the second end portion 173 of the textured layer 166 and the outflow end portion 103 of the frame 106 to each other. The shorter axial distance between the second end portion 173 of the textured layer 166 and the outflow end portion 103 of the frame 106 allows the second end portion 173 of the textured layer 166 to extend radially away from the frame 106 in a gradual manner commensurate with the frame's foreshortening. In some examples, when the frame 106 is fully expanded to its final desired diameter inside the native heart valve 20, the axial suture 183 is long enough to allow the textured layer to fully unfold and axially extend in a distal direction following foreshortening of the frame, as schematically illustrated in FIG. 8B.

In some examples, when the frame 106 is fully expanded to its final desired diameter inside the native heart valve 20, the second portion 178 is not necessarily fully extended in the axial direction, but may be also oriented to some extent radially away, such that the second free end 174 can be positioned radially away from the first free end 172 of the textured layer 166 in the unfolded configuration of the outer skirt 140, as schematically illustrated in FIG. 8C. This may result from an axial suture 183 which is not long enough to allow the second portion 178 of the textured skirt 166 to fully flip over. In such examples, the second portion 178 of the textured layer 166 can form a radial bulge at the expense of the length of the landing zone of the textured skirt (i.e., the axial length of the textured skirt's area configured to face the annulus and native leaflets). Such radial bulging of the textured skirt 166 can assist in improved sealing against the native heart valve 20.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:
a frame movable between a radially compressed state and a radially expanded state, the frame extending between an inflow end and an outflow end, and defining a frame outer surface;
an outer skirt comprising:
   a base layer defining a first surface and a second surface, the base layer comprising:
      a first portion extending distally from a first free end of the base layer; and
      a second portion extending distally from the first portion of the base layer; and
   a textured layer defining a first surface and a second surface, the textured layer comprising:
      a first portion extending distally from a first free end of the textured layer to an intermediate portion; and
      a second portion extending between the intermediate portion and a second free end of the textured layer;
wherein the first portion of the base layer is attached to the frame;
wherein the first portion of the textured layer is attached to the first portion of the base layer; and
wherein the outer skirt is movable between a folded configuration in which the second portion of the textured layer is folded over the first portion of the textured layer, and an unfolded configuration in which the second portion of the textured layer extends distally from the first portion of the textured layer.

Example 2. The prosthetic valve of any example herein, particularly example 1, wherein the second free end of the textured layer is oriented towards the outflow end of the frame in the folded configuration, and is oriented towards the inflow end of the frame in the unfolded configuration.

Example 3. The prosthetic valve of any example herein, particularly example 1 or 2, wherein the second free end of the textured layer is closer to the outflow end of the frame in the folded configuration than in the unfolded configuration.

Example 4. The prosthetic valve of any example herein, particularly any one of examples 1 to 3, wherein the second free end of the textured layer is closer to the first free end of the base layer in the folded configuration than in the unfolded configuration.

Example 5. The prosthetic valve of any example herein, particularly any one of examples 1 to 4, wherein the second portion of the textured layer is not attached to the second portion of the base layer.

Example 6. The prosthetic valve of any example herein, particularly any one of examples 1 to 5, wherein the second portion of the textured layer is movable between the folded configuration and the unfolded configuration, relative to the second portion of the base layer.

Example 7. The prosthetic valve of any example herein, particularly any one of examples 1 to 6, wherein the intermediate portion of the textured layer is not attached to the base layer.

Example 8. The prosthetic valve of any example herein, particularly any one of examples 1 to 7, wherein the second free end of the textured layer is proximal to the intermediate portion in the folded configuration, and is distal to the intermediate portion in the unfolded configuration.

Example 9. The prosthetic valve of any example herein, particularly any one of examples 1 to 8, wherein the second portion of the textured layer is parallel to, and is positioned radially outwards from, the first portion of the base layer in the folded configuration.

Example 10. The prosthetic valve of any example herein, particularly any one of examples 1 to 9, wherein the second portion of the textured layer is parallel to, and is positioned radially outwards from, the second portion of the base layer in the folded configuration.

Example 11. The prosthetic valve of any example herein, particularly any one of examples 1 to 10, wherein the intermediate portion of the textured layer forms an intermediate fold in the folded configuration.

Example 12. The prosthetic valve of any example herein, particularly any one of examples 1 to 11, wherein the first portion of the base layer is disposed around an outer surface of the frame.

Example 13. The prosthetic valve of any example herein, particularly any one of examples 1 to 12, wherein the second portion of the base layer is disposed around the frame outer surface.

Example 14. The prosthetic valve of any example herein, particularly any one of examples 1 to 13, wherein the first portion of the base layer is sutured to the frame.

Example 15. The prosthetic valve of any example herein, particularly any one of examples 1 to 14, wherein the second portion of the base layer is attached to the frame.

Example 16. The prosthetic valve of any example herein, particularly any one of examples 1 to 15, wherein the second portion of the base layer is sutured to the frame.

Example 17. The prosthetic valve of any example herein, particularly any one of examples 1 to 16, wherein the first surface along the first portion of the base layer is facing the frame outer surface.

Example 18. The prosthetic valve of any example herein, particularly any one of examples 1 to 17, wherein the first surface along the first portion of the base layer is in contact with the frame outer surface.

Example 19. The prosthetic valve of any example herein, particularly any one of examples 1 to 18, wherein the first surface along the first portion of the textured layer is facing the second surface along the first portion of the base layer.

Example 20. The prosthetic valve of any example herein, particularly any one of examples 1 to 19, wherein the first surface along the first portion of the textured layer is in contact with the second surface along the first portion of the base layer.

Example 21. The prosthetic valve of any example herein, particularly any one of examples 1 to 20, wherein the second surface along the second portion of the textured layer is facing the first surface along the first portion of the textured layer in the folded configuration.

Example 22. The prosthetic valve of any example herein, particularly any one of examples 1 to 21, wherein the second surface along the second portion of the textured layer is oriented towards the frame in the folded configuration.

Example 23. The prosthetic valve of any example herein, particularly any one of examples 1 to 22, wherein the second surface along the second portion of the textured layer is oriented radially inwards in the folded configuration.

Example 24. The prosthetic valve of any example herein, particularly any one of examples 1 to 23, wherein the second surface along the second portion of the textured layer is oriented away from the frame in the unfolded configuration.

Example 25. The prosthetic valve of any example herein, particularly any one of examples 1 to 24, wherein the second surface along the second portion of the textured layer is oriented radially outwards in the unfolded configuration.

Example 26. The prosthetic valve of any example herein, particularly any one of examples 1 to 25 wherein the textured layer comprises a plurality of radially extending yarns.

Example 27. The prosthetic valve of example 26 wherein the yarns extend away from the second surface of the textured layer.

Example 28. The prosthetic valve of any example herein, particularly example 26 or 27, wherein the yarns comprise first yarns extending away from the first portion of the textured layer, and second yarns extending away from the second portion of the textured layer.

Example 29. The prosthetic valve of any example herein, particularly example 28, wherein the first yarns extend away from the frame.

Example 30. The prosthetic valve of any example herein, particularly example 28 or 29, wherein the second yarns extend towards the first yarns in the folded configuration, and extend away from the frame in the unfolded configuration.

Example 31. The prosthetic valve of any example herein, particularly any one of examples 1 to 30, wherein the first portion of the textured layer is bonded to the first portion of the base layer.

Example 32. The prosthetic valve of any example herein, particularly any one of examples 1 to 30, wherein the first portion of the textured layer is releasably attached to the first portion of the base layer.

Example 33. The prosthetic valve of any example herein, particularly example 32, further comprising at least one retaining suture and at least one pull suture, the at least one retaining suture forming a plurality of whip stitches extending around at least one pull suture and through the textured layer and the base layer, wherein the at least one pull suture is configured to be pulled through and away from the whip stitches, thereby detaching the textured layer from the base layer.

Example 34. The prosthetic valve of any example herein, particularly example 33, wherein each whip stitch is threaded through a single aperture in the textured layer and two adjacent apertures in the base layer.

Example 35. The prosthetic valve any example herein, particularly example 33 or 34, wherein the at least one pull suture comprises a first pull suture circumferentially extending along a portion of the textured layer which is closer to the first free end of the textured layer, and a second pull suture circumferentially extending along a portion of the textured layer which is closer to the intermediate portion of the textured layer.

Example 36. The prosthetic valve of any example herein, particularly any one of examples 1 to 35, wherein the base layer has a greater thromboresistance than the textured layer.

Example 37. The prosthetic valve of any example herein, particularly any one of examples 1 to 36, wherein the base layer comprises a polymeric material.

Example 38. The prosthetic valve of any example herein, particularly example 37, wherein the polymeric material is thermoplastic polyurethane.

Example 39. The prosthetic valve of any example herein, particularly any one of examples 1 to 38, wherein the base layer further comprises a third portion folded over the second portion of the base layer, and a fourth portion extending between the third portion of the base layer and a second free end of the base layer.

Example 40. The prosthetic valve of any example herein, particularly example 39, wherein the base layer further comprises an inflow fold defined between the second portion and the third portion of the base layer.

Example 41. The prosthetic valve of any example herein, particularly example 40, wherein an inflow fold portion of the base layer, extending from the inflow fold of the base layer, is folded around the inflow end of the frame.

Example 42. The prosthetic valve of any example herein, particularly any one of examples 39 to 41, wherein the base layer further defines a dividing fold between the third portion and the fourth portion of the base layer, and wherein the dividing fold defines an overfold in the unfolded configuration.

Example 43. The prosthetic valve of any example herein, particularly example 42, wherein the dividing fold is positioned adjacent to the intermediate portion of the textured layer.

Example 44. The prosthetic valve of any example herein, particularly any one of examples 39 to 43, wherein the fourth portion of the base layer is parallel to, and is positioned radially outwards from, the first portion of the base layer in the folded configuration.

Example 45. The prosthetic valve of any example herein, particularly any one of examples 39 to 44, wherein the fourth portion of the base layer is parallel to, and is positioned radially outwards from, the third portion of the base layer in the unfolded configuration.

Example 46. The prosthetic valve of any example herein, particularly any one of examples 39 to 45, wherein the second free end of the base layer is oriented towards the outflow end of the frame in the folded configuration, and is oriented towards the inflow end of the frame in the unfolded configuration.

Example 47. The prosthetic valve of any example herein, particularly any one of examples 39 to 46, wherein the second free end of the base layer is closer to the outflow end of the frame in the folded configuration than in the unfolded configuration.

Example 48. The prosthetic valve of any example herein, particularly any one of examples 39 to 47, wherein the second free end of the base layer is closer to the first free end of the base layer in the folded configuration than in the unfolded configuration.

Example 49. The prosthetic valve of any example herein, particularly any one of examples 39 to 48, wherein the fourth portion of the base layer is movable between the folded configuration and the unfolded configuration, relative to the third portion of the base layer.

Example 50. The prosthetic valve of any example herein, particularly any one of examples 39 to 49, wherein second free end of the base layer is proximal to the intermediate portion of the textured layer in the folded configuration, and is distal to the intermediate portion of the textured layer in the unfolded configuration.

Example 51. The prosthetic valve of any example herein, particularly any one of examples 39 to 50, wherein the third portion of the base layer is attached to the second portion of the base layer.

Example 52. The prosthetic valve of any example herein, particularly any one of examples 39 to 51, wherein the second surface along the third portion of the base layer is facing the second surface along the second portion of the base layer.

Example 53. The prosthetic valve of any example herein, particularly any one of examples 39 to 52, wherein the second surface along the third portion of the base layer is in contact with the second surface along the second portion of the base layer.

Example 54. The prosthetic valve of any example herein, particularly any one of examples 39 to 53, wherein the first surface along the second portion of the textured layer is facing the second surface along the fourth portion of the base layer.

Example 55. The prosthetic valve of any example herein, particularly any one of examples 39 to 54, wherein the first surface along the second portion of the textured layer is in contact with the second surface along the fourth portion of the base layer.

Example 56. The prosthetic valve of any example herein, particularly any one of examples 39 to 55, wherein the first surface along the fourth portion of the base layer is oriented away from the frame in the folded configuration.

Example 57. The prosthetic valve of any example herein, particularly any one of examples 39 to 56, wherein the first surface along the fourth portion of the base layer is oriented radially outwards in the folded configuration.

Example 58. The prosthetic valve of any example herein, particularly any one of examples 39 to 57, wherein the first surface along the fourth portion of the base layer is oriented towards the frame in the unfolded configuration.

Example 59. The prosthetic valve of any example herein, particularly any one of examples 39 to 58, wherein the first surface along the fourth portion of the base layer is oriented radially inwards in the unfolded configuration.

Example 60. The prosthetic valve of any example herein, particularly any one of examples 39 to 59, wherein the fourth portion of the base layer is positioned radially outwards to the second portion of the textured layer in the folded configuration.

Example 61. The prosthetic valve of any example herein, particularly any one of examples 39 to 60, wherein the fourth portion of the base layer is positioned radially inwards to the second portion of the textured layer in the unfolded configuration.

Example 62. The prosthetic valve of any example herein, particularly any one of examples 39 to 61, wherein the textured layer is disposed between the first portion of the base layer and the fourth portion of the base layer in the folded configuration.

Example 63. The prosthetic valve of any example herein, particularly any one of examples 39 to 62, wherein the textured layer is positioned radially outwards to the base layer in the unfolded configuration.

Example 64. The prosthetic valve of any example herein, particularly any one of examples 39 to 63, wherein the second surface of the textured layer is oriented radially outwards from the frame in the unfolded configuration.

Example 65. The prosthetic valve of any example herein, particularly any one of examples 39 to 64, wherein the textured layer is separated from the frame by the base layer.

Example 66. The prosthetic valve of any example herein, particularly any one of examples 39 to 65, further comprising at least one axial suture extending between an outflow end portion of the frame and a second end portion of the textured layer.

Example 67. The prosthetic valve of any example herein, particularly example 66, wherein the at least one axial suture is taut between the outflow end portion of the frame and the second end portion of the textured layer, in the folded configuration.

Example 68. The prosthetic valve of any example herein, particularly example 66 or 67, wherein the second free end of the textured layer is positioned radially outwards relative to the first free end of the textured layer, in the unfolded configuration.

Example 69. The prosthetic valve of any example herein, particularly any one of examples 66 to 68, wherein the second free end of the textured layer is proximal to the inflow end of the frame in the unfolded configuration.

Example 70. The prosthetic valve of any example herein, particularly any one of examples 1 to 69, further comprising a valvular structure mounted within the frame and configured to regulate flow of blood through the frame in one direction.

Example 71. A method of implanting a prosthetic valve, comprising:
advancing a delivery assembly that comprises a delivery apparatus carrying the prosthetic valve retained in a radially compressed state inside an outer shaft the delivery apparatus, to a native heart valve, wherein the prosthetic valve comprises an outer skirt retained in a folded configuration thereof inside the outer shaft;
deploying the prosthetic valve out of the outer shaft;
positioning a second free end of a textured layer of the outer skirt distal to native leaflets of the native heart valve; and
moving the outer skirt to an unfolded configuration thereof by expanding the prosthetic valve such that the second free end of a textured layer is distally pushed by the native leaflets.

Example 72. The method of any example herein, particularly example 71, wherein the outer skirt comprises a base layer attached to a frame of the prosthetic valve, wherein a first portion of the textured layer is attached to a first portion of the base layer, and wherein a second portion of the textured layer is movable relative to the frame between the folded configuration and the unfolded configuration.

Example 73. The method of any example herein, particularly example 72, wherein the textured layer is separated from the frame by the base layer.

Example 74. The method of any example herein, particularly example 72 or 73, wherein the second portion of the textured layer is folded over the first portion of the textured layer in the folded configuration, and wherein the moving the outer skirt to the unfolded configuration comprises unfolding the textured layer.

Example 75. The method of any example herein, particularly any one of examples 72 to 74, wherein a second surface of the textured layer is concealed from the outer shaft in the folded configuration.

Example 76. The method of any example herein, particularly example 75, wherein the moving the outer skirt to the unfolded configuration comprises exposing the second surface of the textured layer to the native heart valve.

Example 77. The method of any example herein, particularly example 75 or 76, wherein the textured layer further comprises a plurality of yarns extending away from the second surface of the textured layer.

Example 78. The method of any example herein, particularly example 77, wherein the yarns are concealed from the outer shaft in the folded configuration.

Example 79. The method of any example herein, particularly example 77 or 78, wherein the moving the outer skirt to the unfolded configuration comprises exposing the yarns to the native heart valve.

Example 80. The method of any example herein, particularly any one of examples 71 to 79, wherein the positioning the second free end of the textured layer distal to the native leaflets comprises approximating the second free end of the textured layer to the native leaflets.

Example 81. The method of any example herein, particularly any one of examples 71 to 80, wherein the positioning the second free end of the textured layer distal to the native leaflets expanding the prosthetic valve comprises inflating a balloon around which the prosthetic valve is crimped.

Example 82. The method of any example herein, particularly example 81, further comprising deflating the balloon and retrieving the delivery apparatus.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state, the frame extending between an inflow end and an outflow end, and defining a frame outer surface;
an outer skirt comprising:
a base layer defining a first surface and a second surface, the base layer comprising:
a first portion extending distally from a first free end of the base layer; and
a second portion extending distally from the first portion of the base layer; and
a textured layer defining a first surface and a second surface, the textured layer comprising:
a first portion extending distally from a first free end of the textured layer to an intermediate portion; and
a second portion extending between the intermediate portion and a second free end of the textured layer;
wherein the first portion of the base layer is attached to the frame;
wherein the first portion of the textured layer is attached to the first portion of the base layer; and
wherein the outer skirt is movable between a folded configuration in which the second portion of the textured layer is folded over the first portion of the textured layer, and an unfolded configuration in which the second portion of the textured layer extends distally from the first portion of the textured layer.

2. The prosthetic valve of claim 1, wherein the second free end of the textured layer is oriented towards the outflow end of the frame in the folded configuration, and is oriented towards the inflow end of the frame in the unfolded configuration.

3. The prosthetic valve of claim 1, wherein the second free end of the textured layer is closer to the outflow end of the frame in the folded configuration than in the unfolded configuration.

4. The prosthetic valve of claim 1, wherein the second surface along the second portion of the textured layer is facing the first surface along the first portion of the textured layer in the folded configuration.

5. The prosthetic valve of claim 1, wherein the second surface along the second portion of the textured layer is oriented towards the frame in the folded configuration.

6. The prosthetic valve of claim 1, wherein the second surface along the second portion of the textured layer is oriented away from the frame in the unfolded configuration.

7. The prosthetic valve of claim 1, wherein the second surface along the second portion of the textured layer is oriented radially outwards in the unfolded configuration.

8. The prosthetic valve of claim 1, wherein the textured layer comprises a plurality of radially extending yarns.

9. The prosthetic valve of claim 8, wherein the yarns comprise first yarns extending away from the first portion of the textured layer, and second yarns extending away from the second portion of the textured layer.

10. The prosthetic valve of claim 9, wherein the second yarns extend towards the first yarns in the folded configuration, and extend away from the frame in the unfolded configuration.

11. The prosthetic valve of claim 1, wherein the first portion of the textured layer is releasably attached to the first portion of the base layer.

12. The prosthetic valve of claim 11, further comprising at least one retaining suture and at least one pull suture, the at least one retaining suture forming a plurality of whip stitches extending around at least one pull suture and through the textured layer and the base layer, wherein the at least one pull suture is configured to be pulled through and away from the whip stitches, thereby detaching the textured layer from the base layer.

13. The prosthetic valve of claim 1, wherein the base layer further comprises a third portion folded over the second portion of the base layer, and a fourth portion extending between the third portion of the base layer and a second free end of the base layer.

14. The prosthetic valve of claim 1, further comprising at least one axial suture which is taut between an outflow end portion of the frame and a second end portion of the textured layer, in the folded configuration.

15. A method of implanting a prosthetic valve, comprising:
advancing a delivery assembly that comprises a delivery apparatus carrying the prosthetic valve retained in a radially compressed state inside an outer shaft the delivery apparatus, to a native heart valve, wherein the prosthetic valve comprises an outer skirt retained in a folded configuration thereof inside the outer shaft;
deploying the prosthetic valve out of the outer shaft;
positioning a second free end of a textured layer of the outer skirt distal to native leaflets of the native heart valve; and
moving the outer skirt to an unfolded configuration thereof by expanding the prosthetic valve such that the second free end of a textured layer is distally pushed by the native leaflets.

16. The method of claim 15, wherein the outer skirt comprises a base layer attached to a frame of the prosthetic valve, wherein a first portion of the textured layer is attached to a first portion of the base layer, and wherein a second portion of the textured layer is movable relative to the frame between the folded configuration and the unfolded configuration.

17. The method of claim 16, wherein the second portion of the textured layer is folded over the first portion of the textured layer in the folded configuration, and wherein the moving the outer skirt to the unfolded configuration comprises unfolding the textured layer.

18. The method of claim 16, wherein the second surface of the textured layer is concealed from the outer shaft in the folded configuration, and wherein the textured layer further comprises a plurality of yarns extending away from the second surface of the textured layer.

19. The method of claim 18, wherein the yarns are concealed from inner walls of the outer shaft in the folded configuration.

20. The method of claim 18, wherein the moving the outer skirt to the unfolded configuration comprises exposing the yarns to the native heart valve.
